(19) United States
(12) Patent Application Publication
(10) Pub. No.: US 2015/0374245 A1
(43) Pub. Date: Dec. 31, 2015

(54) MONITORING ARTERIAL BLOOD FLOW

(71) Applicant: Heptagon Micro Optics Pte. Ltd., Singapore (SG)

(72) Inventor: Andrei Szilagyi, Rancho Palos Verdes, CA (US)

(21) Appl. No.: 14/740,610

(22) Filed: Jun. 16, 2015

Related U.S. Application Data
(60) Provisional application No. 62/017,417, filed on Jun. 26, 2014.

(51) Int. Cl. A61B 5/026; A61B 5/1455; A61B 5/0205; A61B 5/024

(52) U.S. Cl. CPC A61B 5/0261; A61B 5/024; A61B 5/14551; A61B 5/0205

(57) ABSTRACT

A device for monitoring arterial blood flow includes: a carrier substrate positioned on or affixed to a user's body; an array of light emitting elements on the carrier substrate, in which each light emitting element is arranged to emit light into the user when the carrier is positioned on the user's body; an array of light detecting elements on the carrier substrate, in which each light detecting element is arranged to detect light generated by one or more of the light emitting elements after the light has reflected from an object within the user's body; and an electronic controller to discretely activate one or more of the light emitting elements while simultaneously deactivating one or more other light emitting elements in the array, receive a signal generated by at least one light detecting element, and determine, based on the received signal, a condition of the user's body.

### BACKGROUND

When applied to living bodies, Heart Rate Monitoring (HRM) devices that employ optical illumination and detection techniques must contend with light absorption and light scattering phenomena which degrade and diminish the detected signal. To mitigate such losses, HRM devices typically comprise one large and powerful light emitting diode (LED) flanked or surrounded by several photo detectors (PDs) and placed facing against the subject's body. During operation of the HRM device, some of the light emitted from the LED may encounter an arterial vessel where pulsatile blood flow can modulate the absorption of the incident light. Some of the unabsorbed light reflected or scattered from the arterial vessel may reach and be detected by one of the PDs. Based on the change in absorption with time, an estimate of the heart rate may be determined.

### SUMMARY

The present disclosure relates to monitoring arterial blood flow.

In general, in a first aspect, the subject matter disclosed herein may be embodied in devices for monitoring arterial blood flow, in which the devices include: a carrier substrate configured to be positioned on or affixed to a user's body; an array of light emitting elements on the carrier substrate, in which each light emitting element in the array is arranged to emit light into the user when the carrier is positioned on or affixed to the user's body; an array of light detecting elements on the carrier substrate, in which each light detecting element is arranged to detect light generated by one or more of the light emitting elements after the light has reflected from an object within the user's body; and an electronic controller configured to: discretely activate one or more of the light emitting elements while simultaneously deactivating one or more other light emitting elements in the array, receive a signal generated by at least one light detecting element in response to detecting light, and determine, based on the received signal, a condition of the user's body.

Implementations of the devices may include one or more of the following features and/or features of other aspects. For example, in some implementations, the condition is a heart rate of the user.

In some implementations, the condition is a blood oxygenation level of the user.

In some implementations, the light emitting elements are arranged in a row with approximately equal spacing between adjacent light emitting elements, and the light detecting elements are arranged in a row with approximately equal spacing between adjacent light detecting elements, with the rows approximately parallel to, and laterally offset from each other. The row of light emitting elements and the row of light detecting elements may be offset both laterally and longitudinally from each other.

In some implementations, the carrier includes a watchband.

In some implementations, the electronic controller includes a power supply, and an electronic processor electronically coupled to the array of light emitting elements and to the array of light detecting elements.

In some implementations, each light detecting element in the array of light detecting elements is aligned with a corresponding light emitting element in the array of light emitting elements, such that each light detecting element is closest to the corresponding light emitting element.

In some implementations, the array of light emitting elements is laterally and longitudinally offset from the array of light detecting elements, such that each light detecting element is positioned approximately equidistant from two light emitting elements.

In some implementations, the array of light emitting elements includes two or more rows of light emitting elements.

In some implementations, the array of light detecting elements includes two or more rows of light detecting elements.

In some implementations, the array of light emitting elements includes two or more rows of light emitting elements, and the array of light detecting elements includes two or more rows of light detecting elements. Each light emitting element in a first row of light emitting elements nay be aligned with a corresponding light detecting element in a first row of light detecting elements to form a first emitter-detector row pair. Each light emitting element in a second row of light emitting elements may be aligned with a corresponding light detecting element in a second row of light detecting elements to form a second emitter-detector row pair. The first emitter-detector row pair may be laterally and longitudinally offset from the second emitter-detector row pair. The lateral offset distance may be equal to one half of a pitch between adjacent light emitting elements in the first or second row of light emitting elements or one half of a pitch between adjacent light detecting elements in the first or second row of light detecting elements. The number of light emitting elements in the first row of light emitting elements may equal the number of light emitting elements in the second row of light emitting elements. Each light emitting element in the first row of light emitting elements may be aligned with a corresponding light emitting element in the second row of light emitting elements.

In some implementations, the array of light emitting elements is configured to emit light at a first wavelength and a second different wavelength. Each light emitting element in a first row of light emitting elements may be configured to emit light at the first wavelength, and each light emitting element the second row of light emitting elements may be configured to emit light at the second wavelength. The array of light detecting elements may be arranged midway between the first row of light emitting elements and the second row of light emitting elements.

In another aspect, the subject matter of the present disclosure may be embodied in methods of monitoring a condition in a user, in which the methods include positioning a device onto a limb of the user, in which the device includes: a carrier substrate configured to be positioned on or affixed to a user's body; an array of light emitting elements on the carrier substrate, in which each light emitting element in the array is arranged to emit light into the user when the carrier is positioned on or affixed to the user's body; an array of light detecting elements on the carrier substrate, in which each light detecting element is arranged to detect light generated by one or more of the light emitting elements after the light has reflected from an object within the user's body; and an electronic controller configured to: discretely activate one or more of the light emitting elements while simultaneously deactivating one or more other light emitting elements in the array, receive a signal generated by at least one light detecting element in response to detecting light, and determine, based on the received signal, a condition of the user's body. The method further includes: b) sequentially activating the light emitting elements; c) identifying at least one emitter-detector pair that provides the highest signal to noise ratio; d) deactivating all of the light emitting elements and light detecting elements other than said at least one emitter-detector pair that provides the highest signal to noise ratio.

Implementations of the methods may include one or more of the following features. For example, the methods may further include receiving an electronic signal from the light detecting element of the emitter-detector pair providing the highest signal to noise ratio; and determining, based on the electronic signal, the condition of the user The condition may be a blood oxygenation level of the user. The condition may be a heart rate of the user.

In some implementations, the methods further include determining whether a trigger event has occurred, and when a trigger event has occurred, repeating a) through d) of the method described above. The trigger event may include determining that the device is within a predefined distance of the user. The trigger event may include determining that the user is no longer within a predefined distance to the device. The trigger event may include being unable to measure a blood pulse signal for a predefined length of time. The trigger event may include a mechanical shock exceeding a pre-defined amplitude.

In another aspect, the subject matter of the present disclosure may be embodied in apparatuses for monitoring a condition of a user, in which an apparatus includes a carrier configured to be positioned on or affixed to the user; a first array of light emitting elements held in place by the carrier, in which each light emitting element in the first array is arranged to emit light toward or into the user when the carrier is positioned on or affixed to the user; a first array of light detecting elements held in place by the carrier, in which each light detecting element in the first array is arranged to receive light that has passed through at least a portion of the user when the carrier is positioned on or affixed to the user; a second array of light emitting elements held in place by the carrier, in which each light emitting element in the second array is arranged to emit light toward or into the user when the carrier is positioned on or affixed to the user; a second array of light detecting elements held in place by the carrier, in which each light detecting element in the second array is arranged to receive light that has passed through at least a portion of the user when the carrier is positioned on or affixed to the user; an electronic controller configured to: discretely activate the light emitting elements in the first and second arrays of light emitting elements, receive electronic signals from the light detecting elements in the first and second arrays of light detecting elements, and determine, based on the electronic signals received from the light detecting elements, the condition of the user.

Implementations of the apparatuses may include one or more of the following features. For example, the electronic controller may be further configured to: identify a pulse propagating through the user; determine a time the pulse takes to travel a predefined distance; reject the pulse as a non-cardiac induced pulse when the time is outside of a predefined range; accept the pulse as a cardiac induced pulse when the time is within the predefined range. The electronic controller may be further configured to determine the condition of the user based only on pulses accepted as cardiac induced pulses. The condition may be a heart rate of the user. The condition may be a blood oxygenation level of the user.

In another aspect, the subject matter of the present disclosure may be embodied in methods of determining a condition of a user, in which the methods include: a) positioning an apparatus onto a limb of the user. The apparatus may include: a carrier configured to be positioned on or affixed to the user; a first array of light emitting elements held in place by the carrier, in which each light emitting element in the first array is arranged to emit light toward or into the user when the carrier is positioned on or affixed to the user; a first array of light detecting elements held in place by the carrier, in which each light detecting element in the first array is arranged to receive light that has passed through at least a portion of the user when the carrier is positioned on or affixed to the user; a second array of light emitting elements held in place by the carrier, in which each light emitting element in the second array is arranged to emit light toward or into the user when the carrier is positioned on or affixed to the user; a second array of light detecting elements held in place by the carrier, in which each light detecting element in the second array is arranged to receive light that has passed through at least a portion of the user when the carrier is positioned on or affixed to the user; an electronic controller configured to: discretely activate the light emitting elements in the first and second arrays of light emitting elements, receive electronic signals from the light detecting elements in the first and second arrays of light detecting elements, and determine, based on the electronic signals received from the light detecting elements, the condition of the user. The methods further include b) sequentially activating the light emitting elements of the first array of light emitting elements and the second array of light emitting elements; c) identifying at least two emitter-detector pairs that provide the highest signal to noise ratio; and d) disabling all of the light emitting elements and light detecting elements other than the identified emitter-detector pairs.

Implementations of the methods may include one or more of the following features. For example, the methods may include identifying a pulse propagating through the user; determining a time the pulse takes to travel a predefined distance; rejecting the pulse as a non-cardiac induced pulse when the time is outside of a predefined range; and accepting the pulse as a cardiac induced pulse when the time is within the predefined range. The methods may further include determining the condition of the user based only on pulses accepted as cardiac induced pulses. The condition may be a heart rate of the user and/or a blood oxygenation level of the user.

In another aspect, the subject matter of the present disclosure may be embodied in apparatuses for monitoring a condition of a user, in which the apparatuses include: a carrier for placement on or around a user's limb; multiple sensors arranged on the carrier, in which each sensor is configured to detect a radial motion and output a signal corresponding to the detected motion, and in which at least two sensors are arranged on the carrier at diametrically opposite positions on the carrier; and an electronic processor coupled to the plurality of sensors, in which the electronic processor is configured to derive information about a lateral movement of the carrier and information about expansion and/or contraction of the carrier based on the signals output by the multiple sensors, and determine a heart rate of a user based on the derived information.

Implementations of the apparatuses may include one or more of the following features. For example, in some implementations, the multiple sensors may include two pairs of accelerometers, in which the accelerometers in each pair are arranged on the carrier at diametrically opposite positions on the carrier, and the accelerometers are approximately uniformly arranged around the band.

In some implementations, the information about the lateral movement of the carrier is based on a difference between signals obtained from at least two diametrically opposed sensors, and the information about the expansion and/or contraction of the carrier is based on an average of the signals obtained from at least two diametrically opposed sensors.

Implementations disclosed herein can offer several advantages. For example, in some implementations, the devices encompassed by the present disclosure have relatively low power consumption. In some implementations, the devices encompassed by the present disclosure are capable of discriminating between normal cardiac-induced blood pulses and spurious transients, as well as suppressing such spurious signals. By suppressing non-cardiac induced pulses, the device also may provide enhanced protection against errors caused by motion, shock and/or vibration. In some implementations, the light emitting elements of the device are operated periodically, thus reducing the sensitivity of the device to light from external sources. In certain implementations, the devices encompassed by the present disclosure can discriminate between motion due to movement of a subject and motion due to blood flow enabling accurate plethysmography measurements.

The details of one or more implementations are set forth in the accompanying drawings and the description below. Other aspects, features and advantages will be apparent from the description, drawings, and claims.

### BRIEF DESCRIPTION OF THE DRAWINGS

FIG. 1 is a schematic that illustrates an example operation of a HRM device.

FIG. 2 is a schematic that illustrates an example of a HRM device that includes multiple light emitting elements and multiple light detecting elements.

FIG. 3 is a schematic illustrating an HRM device including an electronic controller.

FIG. 4 is a flow chart illustrating an example of a process for operating a HRM device.

FIG. 5 is a schematic that illustrates an example of an HRM device that includes two emitter-detector arrays.

FIG. 6 is a schematic that illustrates an example of a HRM device having an array of light emitters and detectors.

FIG. 7 is a schematic that illustrates an example of a device configured to perform pulse oximetry and HRM.

FIG. 8 is a schematic that illustrates an example of a device for discriminating between cardiac induced and non-cardiac induced pulsations.

FIG. 9 is a plot of detector output voltage versus time as recorded by two different detectors for a pulse traveling toward a subject's extremities and for a pulse traveling from a subject's extremities.

FIG. 10 is a flow chart that depicts a process for performing identification and rejection of spurious non-cardiac induced pulses

FIGS. 11-13 are schematics that illustrate example configurations of light emitting elements and light detecting elements.

FIG. 14A is a schematic that illustrates an approximation of limb expansion due to the effect of a volumetric fluid wave arriving at a cross section of the limb.

FIG. 14B is a schematic illustrating a cross-section of a user's limb over which an example of a device for performing plethysmographic HRM is placed.

FIG. 15 is a schematic that illustrates the cross-section of a limb before and after pure limb movement, without any internal fluid flow through the limb.

FIG. 16 is a schematic that illustrates accelerometers for sensing complex movement as indicated by a limb cross-section before and after a combination of internal fluid flow within the limb and lateral motion of the limb.

### DETAILED DESCRIPTION

Certain limitations may be associated with traditional heart rate monitoring devices. For example, such devices typically operate at high power because the emitted light usually must travel relatively long distances before encountering an arterial vessel where pulsatile blood flow modulates the light absorption. Furthermore, the arterial vessels are interspersed with other tissues (e.g., venous vessels) that do not exhibit modulated light absorption as a result of the pumping action of the heart. Thus, the HRM devices must emit a substantial amount of light over large areas so at least some of the relatively low fraction of emitted light that encounters pulsatile arterial flow is detected by one of the PDs. Additionally, HRM devices operate continuously over long periods of time. The continuous operation of the HRM devices at high power and over large areas leads to excessive power consumption that may limit the operating time of the power source driving the device.

In some cases, errors are also introduced into the signals detected by the HRM device as a result of a user's physical activities, e.g., shifting of the device or arterial vessel with respect to one another. Moreover, the portions of the PD detector areas that do not receive light modulated by the heart may nonetheless be subject to undesirable environmental light as well as electromagnetic interference, both of which diminish the resulting signal to noise ratio and increase the difficulty of providing a meaningful device output.

The subject matter of the present disclosure encompasses devices for measuring, among other things, a subject's heart rate, in which the devices have relatively low power consumption through the discrete activation of one or more light emitting elements and the de-activation of one or more other light emitting elements. Furthermore, the devices encompassed by the present disclosure may be capable of discriminating between normal cardiac-induced blood pulses and spurious transients, as well as suppressing such spurious signals. By suppressing non-cardiac induced pulses, the devices also may provide enhanced protection against errors caused by motion, shock and/or vibration. In addition, in some implementations, the one or more light emitting elements of the device are operated periodically, thus reducing the sensitivity of the device to light from external sources.

### Measuring Heart Rate

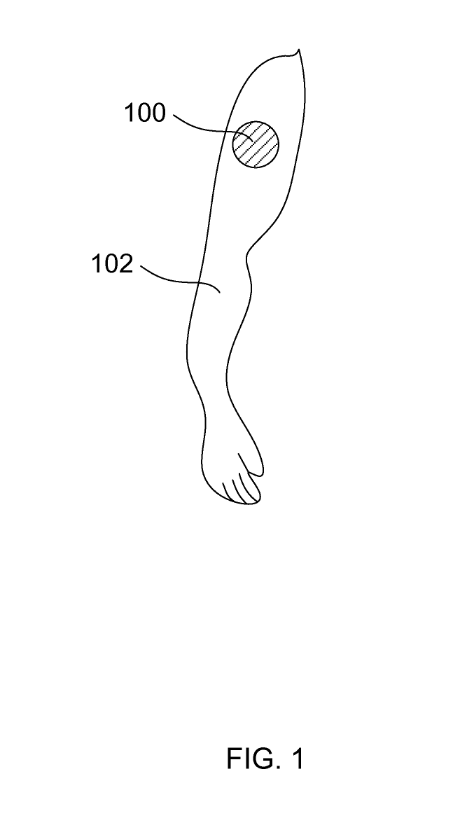

FIG. 1 is a schematic that illustrates an example operation of a HRM device 100 according to the present disclosure. The HRM device 100 is placed against the skin of a subject (e.g., on a limb of a user) 102. During operation, the HRM device 100 emits light that illuminates the skin of the subject 102. A portion of the light passes through the skin into the subcutaneous tissue where it may encounter blood vessels carrying oxygenated arterial blood. With each cardiac cycle, the heart pumps blood through such vessels, causing the blood vessels to expand. The expansion and contraction of the blood vessels and the variation in the amount of oxygenated hemoglobin with each cycle modulates the light reaching the HRM device 100. By monitoring the time-varying change in the amount of light reflected back toward the HRM device 100, it is possible to calculate the corresponding heart rate of the subject 102. The HRM device 100 may include a carrier substrate such as an adhesive pad so that the device 100 can be affixed to the subject or it may include a strap, band, bracelet, watchband, tape, or other structure that can be fitted on and/or around a subject's limb. The carrier may be flexible for fitting around the subject. The carrier substrate may include a single contiguous material, similar to a rubber band or may include a clasp for coupling two ends of the carrier together around the subject. The carrier substrate contains the light emitting elements and light detecting elements and associated electronics for performing the heart rate monitoring described below, as well as other operations disclosed herein.

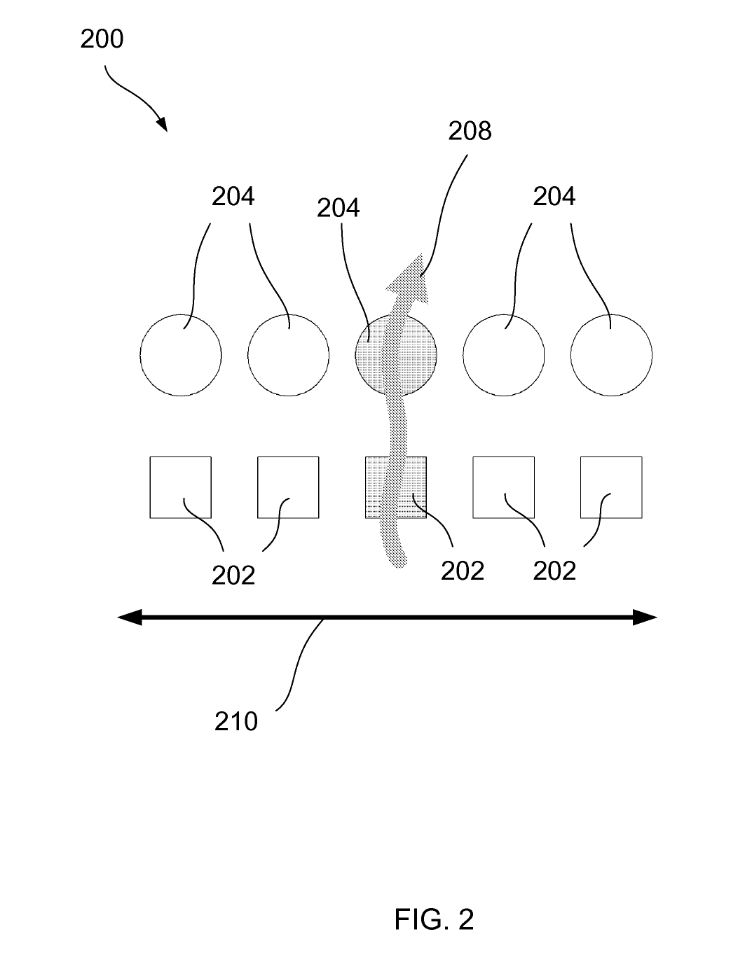

FIG. 2 is a schematic that illustrates an example of a HRM device 200 that includes multiple light emitting elements 202 and multiple light detecting elements 204. As shown in FIG. 2, the light emitting elements 202 are symbolically depicted as squares and the light detecting elements 204 are symbolically depicted as circles. Each light emitting element 202 is aligned with a corresponding light detecting element 204, such that the emitters and detectors are arranged in a linear array. The light emitting elements 202 are arranged in an approximately straight row with approximately equal spacing between adjacent light emitting elements. Similarly, the light detecting elements 204 are arranged in an approximately straight row with approximately equal spacing between adjacent light detecting elements. Appropriate levels of variation in the rows' straightness and in the spacing between elements is allowed depending on the method of construction and/or depending on the desired application. The emitter-detector arrays are arranged on or embedded in the carrier substrate that is positioned on the subject. Examples of light detecting elements (i.e., light detectors) that may be used include, but are not limited to, charge-coupled devices (CCDs), PDs, photoresistors, photovoltaic cells, and/or phototransistors. Examples of light emitting elements (i.e., light emitters) that may be used in the implementation shown in FIG. 1 as well as other implementations include, but are not limited to, LEDs and/or laser diodes, emitting at any appropriate wavelength, such as in the green, red or near infrared.

Vessels carrying oxygenated arterial blood typically have a preferred orientation. For instance, the prevailing orientation of arterial blood vessels in limbs, such as the human arm, is generally parallel to the long dimension of the limb, with the average direction of flow toward the distal regions of the limb. As a result, the pumping action of the heart creates wave-like pressure pulses also propagating toward the outer limb extremities. To improve the detection of the pressure pulses, the emitter-detector arrays are preferably arranged with their elongated dimensions approximately perpendicular to the prevailing arterial flow direction. For instance, as shown in FIG. 2, the arterial flow direction through a blood vessel is represented by the arrow 208 (for ease of viewing, other aspects of the subject are omitted from FIG. 2). The array 200 is positioned above the blood vessel with the long dimension of the array 200 extending approximately perpendicular to the blood flow. Assuming the light emitting surface of the center light emitting element (i.e., the square having the dotted pattern) faces the arterial vessel located beneath the light emitting element 202, a portion of the emitted light then will pass through the skin and impinge on the blood vessel. The center light detecting element (i.e., the circle having the dotted pattern) 204 has its light sensitive surface also facing the subject, and hence the arterial vessel. The center light detecting element 204 then records light that has been modulated by blood in the arterial vessel.

In some implementations, the light emitting element/light detecting element pairs may be discretely activated in succession, thereby resulting in significant power savings. For instance, for an array containing N light emitters and N light detectors, a first light emitting element is activated at a time t₀ to generate incident light. At approximately the same time, the signal received from a corresponding detector is analyzed (e.g., by an electronic processor) to measure light signals that impinge on the detector. All other light emitters and light detectors in the array are deactivated during this measurement. In some implementations, deactivation entails disconnecting electrical power and signal output from the light emitters and/or light detectors. In some implementations, deactivation of the light emitters entails placing the light emitters in a state so that they do not emit any light. In some implementations, the light detectors continue to detect light, but the output signals are not analyzed when the light detectors are deactivated. Subsequently, at time t₁, the next adjacent combination of light emitter and light detector is activated (i.e., the emitter is powered to generate light and the corresponding detector is interrogated to determine a change in measured signal), while all other light emitters and light detectors are deactivated. The process continues until the Nth light emitter and detector are activated at t_{N-1} (and the other light emitters and detectors are deactivated). At this point, sufficient information may have been collected to identify the best emitter/detector pair, i.e., the emitter/detector pair that results in the highest signal-to-noise ratio or that results in a signal-to-noise ratio that exceeds a pre-defined threshold. If necessary, additional scans may be executed, if, for instance, more data averaging is needed for conclusive identification and selection of the best pair.

Not all of the N emitters and detectors need to be activated and analyzed in succession to select a suitable emitter-detector pair. For example, during sequential activation/analysis of the light emitters and light detectors, a threshold signal/value may be observed prior to activating and analyzing one or more remaining emitter-detector combinations in the sequence. In such cases, the emitter-detector pair for which that signal was obtained would be used for further processing, without continuing to scan the output of the remaining emitter-detector combinations. Alternatively, every emitter may be energized simultaneously. However, such implementations may wastefully utilize substantially more power than sequential and discrete activation.

The line 210 in FIG. 2 shows example sequential scan directions of the emitter-detector pairs, i.e., scanning may proceed from left to right or right to left. Other convenient scanning sequences also may be employed. Though the process described above entails activating a single light emitting element and analyzing the output of a single corresponding light detecting element at a time, multiple light detecting elements may be interrogated for each light emitting element that is activated to determine the presence of the pressure pulses. For example, in some implementations, the light emitting elements are individually activated in sequence (i.e., a single light emitting element emits light at any one time) and, during the activation of each light emitter, the outputs of two or more detectors are analyzed to determine whether they are measuring a signal and the value of any such measured signal. In some implementations, the outputs of all the detectors are analyzed for a single activated light emitting element to determine whether a pressure pulse is present.

In some implementations, multiple emitter-detector pairs may be identified as having the desired signal-to-noise ratio or as exceeding a pre-defined threshold. In some cases, the different identified pairs include the same light emitting elements but different light detecting elements. In some cases, the different identified pairs may include different light emitting elements but the same light detecting elements.

In some implementations, the linear emitter-detector array may be oriented in a direction that is approximately perpendicular to the prevailing arterial blood flow. Such an orientation may allow enhanced interception and monitoring of arterial blood flow with fewer emitter/detector pairs.

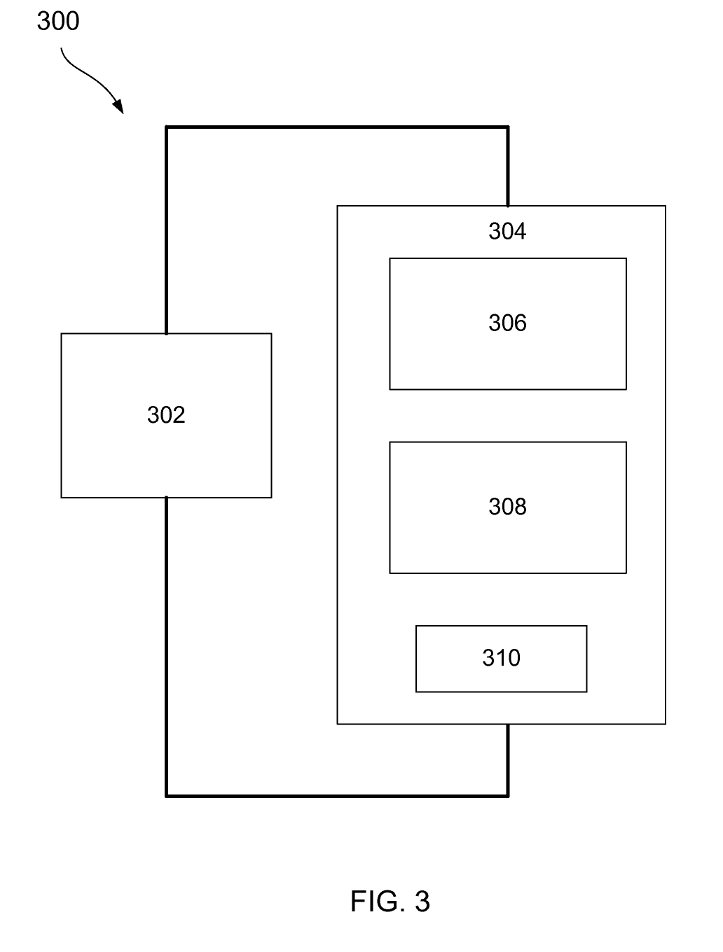

The discrete and sequential activation of the emitters as well as the analysis of the detectors' output is managed by an electronic controller (omitted from FIG. 2 for clarity). FIG. 3 is a schematic illustrating an HRM device 300 that includes a linear emitter-detector array 302, similar to the array shown in FIG. 2, and an electronic controller 304 electronically coupled to the linear array 302. The electronic controller 304 includes an electronic processor 306 (e.g., a microprocessor) that is configured to perform several functions including, but not limited to: controlling the activation of the light emitting elements; receiving measurement signals from the light detecting elements; and processing the received measurement signals to determine a heart rate of the subject. The controller 304 also includes a power supply 308 (e.g., a battery) for providing power to the light emitting elements and detectors of the array. The electronic controller 304 is configured to discretely activate the light emitting elements of the HRM device 300. That is, the controller 304 can activate one or more of the light emitting elements individually or simultaneously for any desired period of time, subject to the timing limits and constraints of the processor 306 and power supply 308. In some implementations, the electronic controller 304 also includes an accelerometer 310 for measuring an acceleration of the HRM device 300, e.g., in case of a sudden movement or physical jolt that causes the HRM device 300 to change position relative to the subject. The acceleration signals measured by the accelerometer 310 are transmitted to the processor 306 for analysis. For instance, the processor 306 may receive a signal from the accelerometer 310 exceeding a predetermined threshold value that indicates a large shock to the HRM device 300, after which the processor 306 may determine that the HRM device 300 needs to be recalibrated, for instance by restarting the electronic scanning of the emitter/detector pairs.

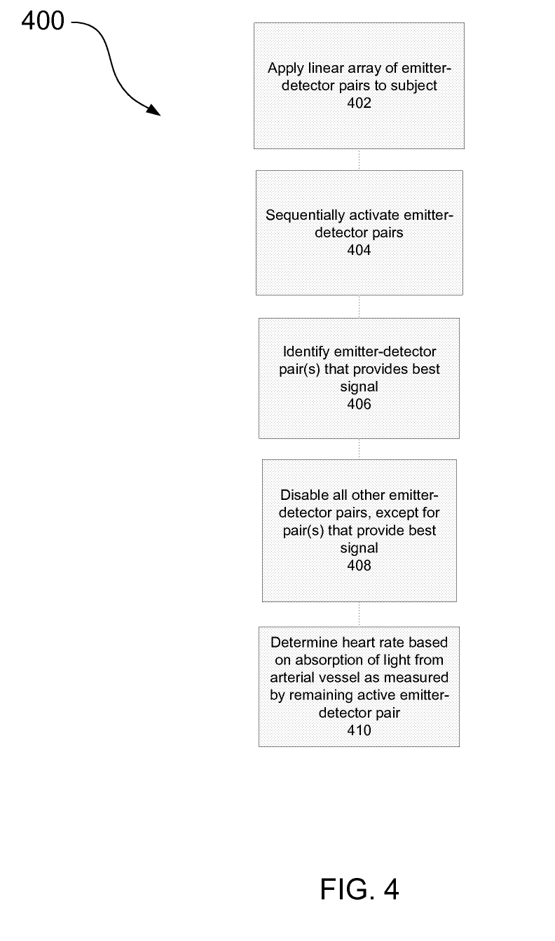

FIG. 4 is a flow chart illustrating an example of a process 400 for operating a HRM device according to the present disclosure. For instance, a linear array of light emitters and light detectors (e.g., LED/PD pairs) is first applied (402) onto a subject's limb. Then, the emitters of the HRM device are sequentially activated and the reflected/scattered light is analyzed (404) (e.g., LED's in the array are sequentially powered while the PD signals are analyzed for indications that a pressure pulse has occurred). Based on the analysis, the emitter-detector pair(s) that provide the best signal source are identified (406). For instance, the best signal source may correspond to the emitter-detector pair that provides the largest signal to noise ratio. In some implementations, multiple emitter-detector pairs are identified as providing the appropriate signal-to-noise ratio. Once the emitter-detector pair(s) that provides the best signal source is identified, all other emitters and detectors are disabled (408). The emitter-detector pair(s) providing the best signal source remain active for measuring the pressure pulses in the arterial vessel. If the active blood vessel passes directly under the space between two adjacent emitter/detector pairs, it may be advantageous to simultaneously select and use both such pairs, while disabling the remaining pairs. Yet another possibility is that an arterial vessel follows a relatively tortuous path while passing under the array. In that case, it may be advantageous, to read not only the detector signal from the pair whose emitter is active, but also the detector signals from pairs adjacent to the active pair. By analyzing signals from the adjacent detectors, it will be possible to make the best choice of which emitter(s) and detector(s) should remain active while the others are disabled. The disabling of the other emitters and detectors effectively reduces the overall power consumption of the HRM device and reduces the electronic noise that may otherwise arise from multiple emitters and detectors being activated. The measurements made by the emitter-detector pair providing the best signal then are transmitted to the electronic controller which determines (410) the heart pulse rate based on the absorption/reflection of the light from the arterial vessel. The computation of the pulse rate may be performed according to any suitable algorithm known in the art, such as described in U.S. Pat. No. 4,063,551, incorporated herein by reference in its entirety.

In some implementations, steps (402)-(406) may be repeated when one or more predefined trigger events occur to reset a baseline. Examples of such pre-defined trigger events include, but are not limited to:
(i) initial detection of proximity to the subject (e.g., a proximity sensor integrated in the HRM device indicates that a suitable object is found within a pre-defined small distance to the proximity sensor); A proximity sensor may include any appropriate proximity sensor, such as, but not limited to, low power capacitive touch sensors, resistive touch sensors, or piezoelectric touch sensors. Detection of proximity to the subject may be understood as occurring when a predefined signal level or predefined change in signal level is detected, depending on the proximity sensor type;
(ii) momentary loss of proximity (e.g., a proximity sensor integrated in the HRM device indicates that no suitable object is found within the pre-defined small distance to itself after the initial detection of the object). Loss of proximity is considered to have occurred when a pre-defined signal level or predefined change in signal level is detected, depending on the proximity sensor type;
(iii) loss of pulsatile signal (occurring, for instance, if the HRM device position has shifted and the previously identified emitter-detector pair(s) no longer correspond to the location of a nearby arterial vessel). The determination of a loss of pulsatile signal may occur when the heart rate analysis algorithm computes a heart rate of zero or close to zero; or
(iv) the detection of a mechanical shock exceeding a predetermined amplitude, for instance a shock of 0.5 g where g is the acceleration due to earth gravity. The shock may be detected, for instance, by an accelerometer integrated in the HRM device).

Since the frequency of heart pulsations is relatively low compared to the speed at which the electronic components of the HRM device can operate, it is possible to electronically scan the entire emitter/detector array multiple times during one cardiac cycle. The scanning would continue for as many heart beats as is necessary to identify the best combination of emitter(s) and detector(s) for measuring pressure pulses as well as the initial heart rate.

Even after the best emitter/detector combination is identified and the other emitters and detectors are temporarily deactivated, sampling may continue with the selected emitter/detector combination. That is, the emitter(s) in the selected combination may be activated in a pulsed manner rather than continuously. The output of the detector(s) from the selected combination may be monitored by the electronic controller continuously or at discrete intervals that synchronously correspond to when the light is output by the emitter(s). Sampling in this manner may have several advantages. For example, in some implementations, performing pulsed sampling with limited duty cycle enables an additional reduction in electrical power consumption over continuous operation of the emitter/detector combination. Furthermore, sampling the output of the detector(s) synchronously with activation of the emitter(s) at a predefined frequency may reduce the background noise associated with undesired environmental light sources, as well as other noise effects.

In some instances, the minimum spacing that can be achieved between emitters, between detectors, and between emitters and detectors is limited as a result of fabrication. Thus, the pitch of the array (e.g., the periodic distance from a point on one emitter-detector pair in the array to a similar point on an adjacent emitter-detector pair in the array) is greater than the width of either a single emitter or a single detector. As a result, adjacent emitters and adjacent detectors cannot be brought arbitrarily close together leaving a gap between adjacent pairs (see, e.g., the space between adjacent emitters and between adjacent detectors in FIG. 2). It is, therefore, possible that the closest measurable arteriole may pass directly under the gap between such pairs, reducing the magnitude of a detected signal.

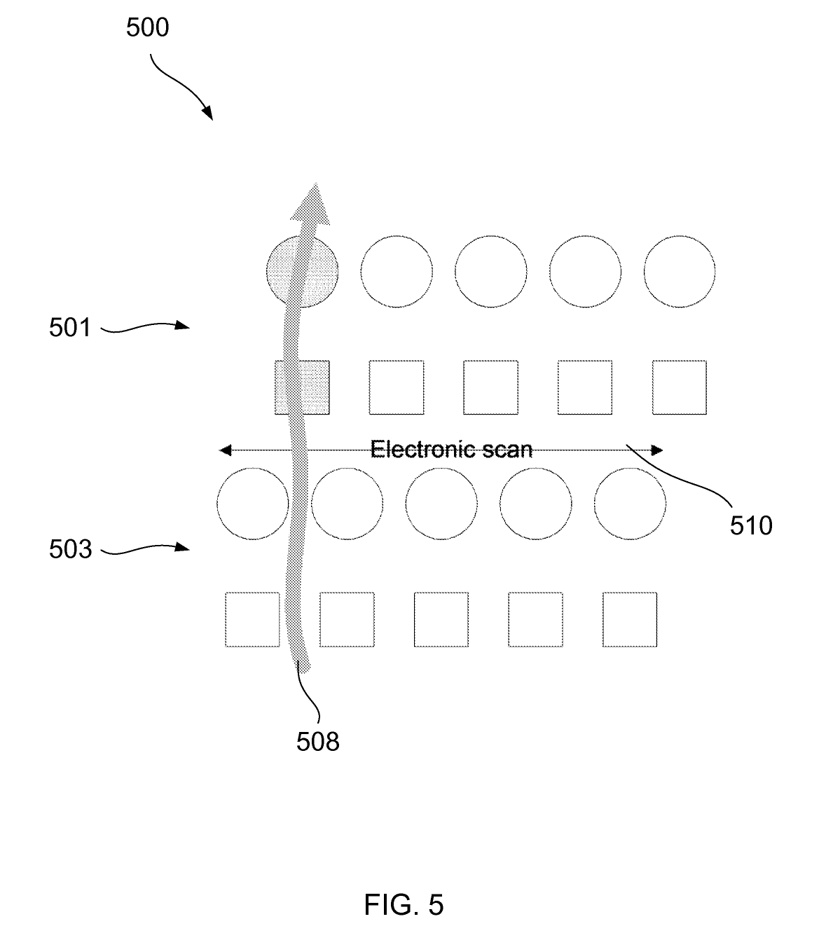

Multiple arrangements are possible to compensate for the possibility that an arterial vessel may pass underneath such gaps. For example, in some implementations, the HRM device may include multiple emitter-detector arrays, in which the arrays are staggered with respect to one another. FIG. 5 is a schematic that illustrates an example of an HRM device 500 that includes two emitter-detector arrays, with the first array 501 laterally offset from the second array 503 (control electronics are emitted for clarity). Specifically, the arrays are arranged such that an emitter-detector pair in the first array 501 is lined up with a gap between adjacent emitter-detector pairs in the second array 503, and vice versa (except for one emitter-detector pair at the end of each array). As in FIG. 2, light emitters are symbolically represented by squares, whereas light detectors are symbolically represented by circles. Thus, a typical blood vessel 508 disposed along a longitudinal direction and traversing the staggered arrays anywhere within the combined lateral extent of the arrays (i.e., the width of the combined arrays along the scan direction) inevitably passes under at least one emitter-detector pair. Accordingly, light from at least one emitter will be modulated by blood in the vessel and be recorded by at least one detector. The light emitters and light detectors may be activated in any sequence similar to those disclosed in relation to FIG. 2. In some implementations, the sequential activation proceeds first through all of the light emitters and light detectors in a first array (e.g., array 501 or array 503) before sequentially activating the light emitters and light detectors of a next array. Alternatively, sequential activation of the light emitters and light detectors may alternate between different arrays (e.g., an emitter-detector pair from the array 501 is activated first, an emitter-detector pair from array 503 is activated second, a different emitter-detector pair from array 501 is activated third, etc.). FIG. 5 shows that the scan direction 510 in which the emitter-detector pairs are activated may proceed left to right or right to left across the arrays.

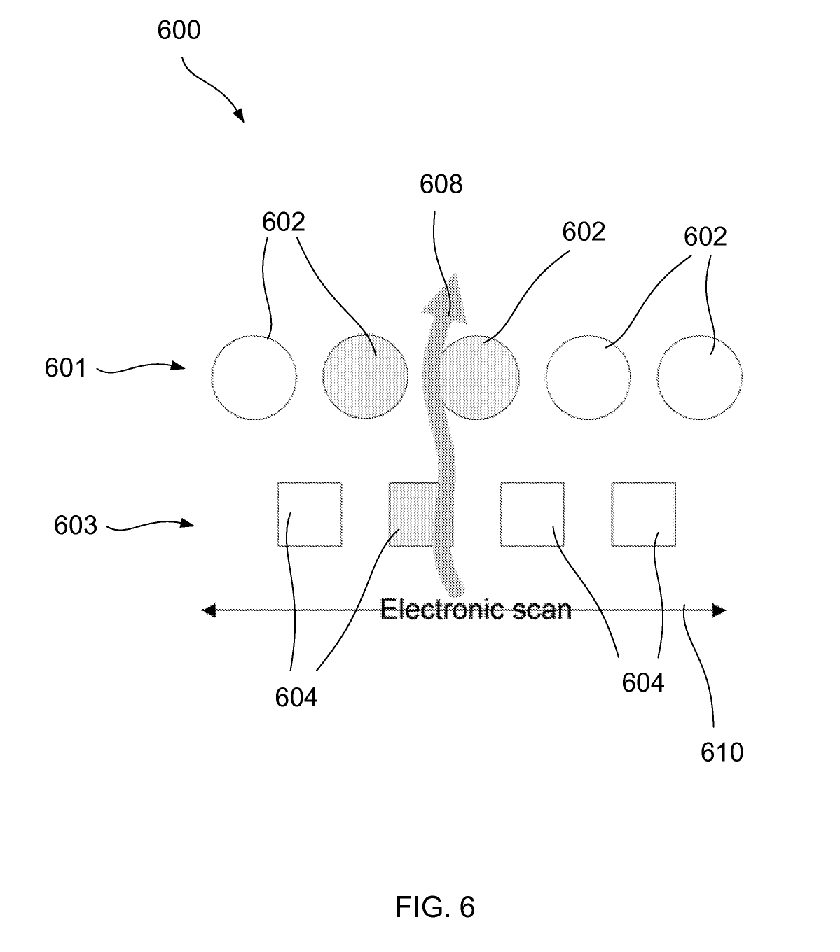

In some implementations, the detectors in the array are offset from the emitters. For instance, FIG. 6 is a schematic that illustrates an example of a HRM device 600 having an array of light emitters and detectors (e.g., LEDs and PDs) in which a row 601 of detectors 602 is offset from a row 603 of emitters 604 by a distance equal to one half the pitch between adjacent detectors/adjacent emitters in the array. In this configuration, typical light detectors can receive tissue-scattered light originating from either of the two nearest LED's. As in other implementations, the light emitting elements may be sequentially activated across the length of the array (scan direction 610) so that at any one point in time, only a single emitter outputs light. Likewise, during the activation of each light emitter, the outputs of one or more detectors (e.g., two, three, four, and so forth) are analyzed to determine whether they are measuring a signal and the value of any such measured signal. A blood vessel 608 passing underneath the array will likely intercept at least a portion of at least one tissue region visited by light travelling between an emitter and an adjacent detector. Such body tissue regions or volumes tend to be configured in the shape of a banana with its narrow ends localized at the emitter and the detector.

### Performing Pulse Oximetry

In some implementations, the device may be configured to operate as a pulsed oximeter as an alternative or in addition to monitoring heart rate. Pulse oximetry is a non-invasive method of monitoring the O₂ saturation in a subject's blood. During pulse oximetry, the subject is illuminated with light having two different wavelengths (e.g., infrared and visible red). As the light passes into the subcutaneous region and is incident on an arterial vessel, the oxygen-rich hemoglobin in the blood absorbs more of the light having the first wavelength and the hemoglobin without oxygen absorbs more of the light having the second wavelength. After absorption, the light is collected by one or more photodetectors sensitive to the wavelengths of interest. A processor (e.g., microprocessor) then determines the differences in absorption and converts the difference into information representative of the amount of oxygen being carried in the blood.

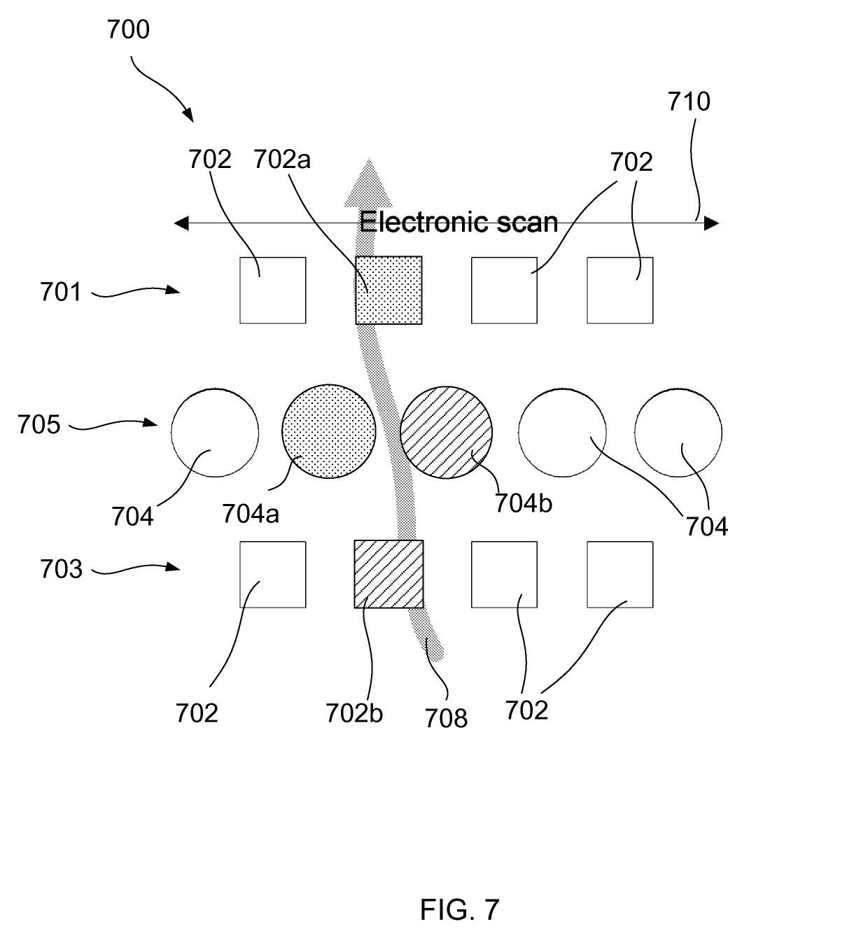

FIG. 7 is a schematic that illustrates an example of a device 700 configured to perform pulse oximetry in addition to heart rate monitoring. The device 700 includes a first row 701 of light emitting elements 702 and a second row 703 of light emitting elements 702, in which row 701 and row 703 are arranged on either side of a row 705 of light detector elements 704. Each emitter 702 in the first row 701 emits light at a first wavelength (e.g., in the infrared portion of the electromagnetic spectrum). Each emitter 702 in the second row 703 emits light at a second different wavelength (e.g., in the red portion of the visible electromagnetic spectrum). Alternatively, rows 701 and 703 may be configured to include both types of emitters, in which the different emitters alternate with one another along the length of the row. Other arrangements are also possible. Each detector 704 may be configured to detect only one of the first or the second wavelength, so that absorption at those wavelengths can be readily determined. Additionally, the detectors 704 may be arranged such that a detector configured to sense light having the first wavelength is adjacent to a detector configured to sense light having the second wavelength, i.e., the wavelength of light sensed by the detectors alternates along the length of the detector array. Alternatively, each of the detectors 704 may be sensitive at both wavelengths, i.e., the detectors have a broad wavelength detection band covering both wavelengths. In this case, the two wavelengths would not be emitted simultaneously. Moreover, identification of which wavelength is being received would be made by time coding, whereby the controlling microprocessor would keep track of which emitter and which detector were being addressed.

During operation of the device 700, light from a first light emitter 702a (e.g., the light emitter having the dot pattern in row 701 of FIG. 7) emits light at a first wavelength towards the subject. A first detector 704a (e.g., the detector having the dot pattern in FIG. 7) senses the light after it has been modulated by blood in an arterial vessel 708 in the subject. As in other implementations, the light emitters in row 701 may be sequentially activated so only a single emitter generates light at a time. Additionally, a second light emitter 702b (e.g., the light emitter having the hatched pattern in row 703 of FIG. 7) emits light at a second wavelength towards the subject. The two wavelengths may be emitted simultaneously if dedicated detectors are used for each, or sequentially if time coding is used. A second detector 704b (e.g., the detector having the hatched pattern in FIG. 7) senses the light after it has been modulated by blood in arterial vessel 708 of the subject. As before, the light emitters in row 703 may be sequentially activated so only a single emitter generates light at a time. In some implementations, using detectors configured to detect only one of the wavelengths may allow better temporal information at the expense of higher peak electrical power from driving two LED's together. In some implementations, using broadband detectors may provide lower peak power and better spatial coverage of the subcutaneous volume under two adjacent detectors, both of which can detect either wavelength, but at the expense of reduced temporal discrimination. The detectors 704 and emitters 702 may be coupled to an electronic controller (excluded from FIG. 7 for clarity), such as the controller shown in FIG. 3, which is configured to determine the amount of absorption at each wavelength and to compute the amount of oxygen in the subject's blood based on the identified absorption. The computation of the oxygen content may be performed according to any suitable algorithm known in the art. The electronic controller also may be configured to determine the subject's heart rate based on the absorption/reflection of a single wavelength, as described above.

In some implementations, the rows of emitter elements are aligned with respect to the row containing the detector elements. For instance, the detectors and emitters may be positioned in a matrix-like arrangement such that each column of the matrix includes two emitters and a detector in a single straight line. Alternatively, as shown in FIG. 7, the first emitter row 701 and/or the second emitter row 703 may be arranged in staggered alignment with respect to the detector row 705 (e.g., each of the first and second rows 701, 703 is displaced along the length of the array with respect to the third row 705 by one half of the array pitch). As a result, a longitudinally disposed arterial blood vessel that extends underneath the device 700 is more likely to pass directly under at least one emitter 702 so as to be exposed to the light generated by that emitter.

As in other implementations, sequential operation of emitter-detector combinations may be performed to determine the emitter-detector combination from each wavelength set that provides the best signal. After identification of the emitter-detector combination that provides the best signal, the less useful emitters and detectors may be selectively turned off, resulting in battery power savings and improved signal to noise ratio. Since, in some cases, the orientation and position of the device with respect to the subject may shift over time or with each new application, the choice of which emitter-detector combinations to use for analysis and which emitters and detectors to deactivate may be made determined by the electronic controller each time the device is mounted on the subject or each time the device is activated to obtain a measurement. This adaptability to changing conditions makes mounting of the device less sensitive to user skill, resulting in improved user experience.

### Differentiating Non-Cardiac Induced Pulse Flows

As previously explained, traditional methods of heart rate monitoring tend to be vulnerable to measurement errors introduced by non-heart related flow transients, e.g., movements of the subject or by other disturbances. Such errors also may arise in pulse oximetry. One method for mitigating such errors is to separate spurious signal contributions traceable to the monolithic movement of blood, non-vascular tissues, and the body from those traceable to blood pulsation. A monolithic movement is one in which either the limb or the blood moves as a block. In actuality, however, spurious signal contributions often result from movements which exhibit wavelike internal degrees of freedom. For instance, the impact of a runner's step initiates a pressure wave which may propagate through the various body tissues at varying speeds reaching upper extremities as a broadened pulse or group of pulses uncorrelated to either heart beating or arm swinging. Some of these pressure waves may even produce reflected waves travelling in the other direction (away from the extremities instead of toward the extremities). Other user motions may also send propagating disturbances away from the extremities. Thus, heart-driven movement of internal fluids or tissues is dynamically distinct from the movements (e.g. positions, velocities or accelerations) that may be detected by external single-point sensors.

These non-heart related flow transients may be distinguished from heart-driven pulses if they have one or more of the following characteristics: 1) if the non-heart related flow transients propagate away from the extremities, i.e., toward the heart; 2) if the non-heart related flow transients propagate outwardly toward the extremities but at a speed substantially different from the pulse induced by the cardiac cycle (e.g., if the pulse frequency is sufficiently fast or slow relative to the cardiac induced pulse such that the difference may be measured); or 3) the non-heart related flow transients include co-propagating venous pulses. Since venous pulses are rich in reduced hemoglobin blood, the presence of a venous pulse can be distinguished from an arterial pulse (which is rich in oxygenated hemoglobin) based on their distinct spectral absorption characteristics when illuminating the different pulses with two distinct wavelengths of light (i.e., a wavelength that is highly absorbed by reduced hemoglobin blood but not oxygenated hemoglobin, and a wavelength that is highly absorbed by oxygenated hemoglobin but not reduced hemoglobin).

Devices for performing HRM and/or pulse oximetry may also be configured to distinguish non-cardiac induced pulses based on the foregoing characteristics. Devices suited for performing such detection are preferably configured to detect rapidly advancing pressure wave fronts. In implementations in which the device is configured to differentiate arterial blood flow from venous blood flow, the device preferably also is configured to detect high concentration of reduced hemoglobin present in venous blood.

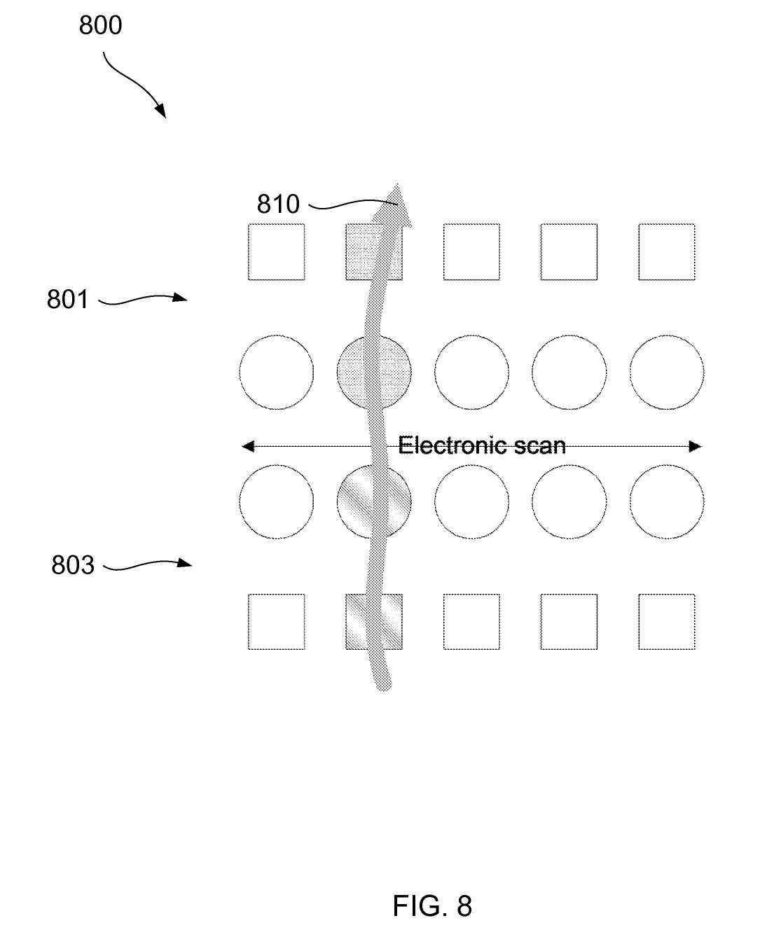

FIG. 8 is a schematic that illustrates an example of a device 800 for discriminating between cardiac induced and non-cardiac induced pulsations. As shown in FIG. 8, the device 800 includes two separate linear arrays (first array 801 and second array 803), each of which includes multiple light emitter-light detector pairs. In the implementation shown in FIG. 8, each emitter-detector pair in the first array 801 is aligned with a corresponding emitter-detector pair in the second array 803, although other configurations are also possible. The emitter-detector pairs of both arrays are electronically coupled to an electronic processor (omitted here for clarity), such as the processor shown in FIG. 3.

In the implementation shown in FIG. 8, a typical arterial blood vessel 810 passes successively underneath two emitter-detector pairs, i.e., one emitter-detector pair from each array. The active emitter-detector pairs in FIG. 8 are shown as being filled with a hatched pattern or being filled with a dot pattern. The emitter-detector pairs from the two arrays may operate at different wavelengths or the same wavelength of light. Blood pressure pulses traveling through such a vessel 810 will therefore be detected by a detector in each array, although their relative time of arrival depends on the speed and direction of the wave. For instance, it is known that the wave velocity in a blood vessel is a nonlinear effect, depending on the blood pressure. Thus, an unusually high amplitude pressure pulse due to, e.g., a mechanical shock, may travel substantially faster through the vascular network than a cardiac induced pulse. Accordingly, by measuring the time it takes the pulse to travel a certain distance (e.g., the distance between detectors), it is possible to differentiate a cardiac induced pulse from an externally induced pulse. If the time in such a case falls below a predetermined threshold, the pulse may be rejected. Likewise, a vigorous movement of a limb may cause simultaneous venous and arterial flow through both emitter-detector arrays, allowing rejection of a non-cardiac induced pulse using the same sub-threshold time difference criterion. Determining the time a pulse takes to travel a fixed distance (and thus determining the speed of the pulse) may be accomplished by identifying when a selected feature of a pulse is recorded by a detector in the first array and a detector in the second array.

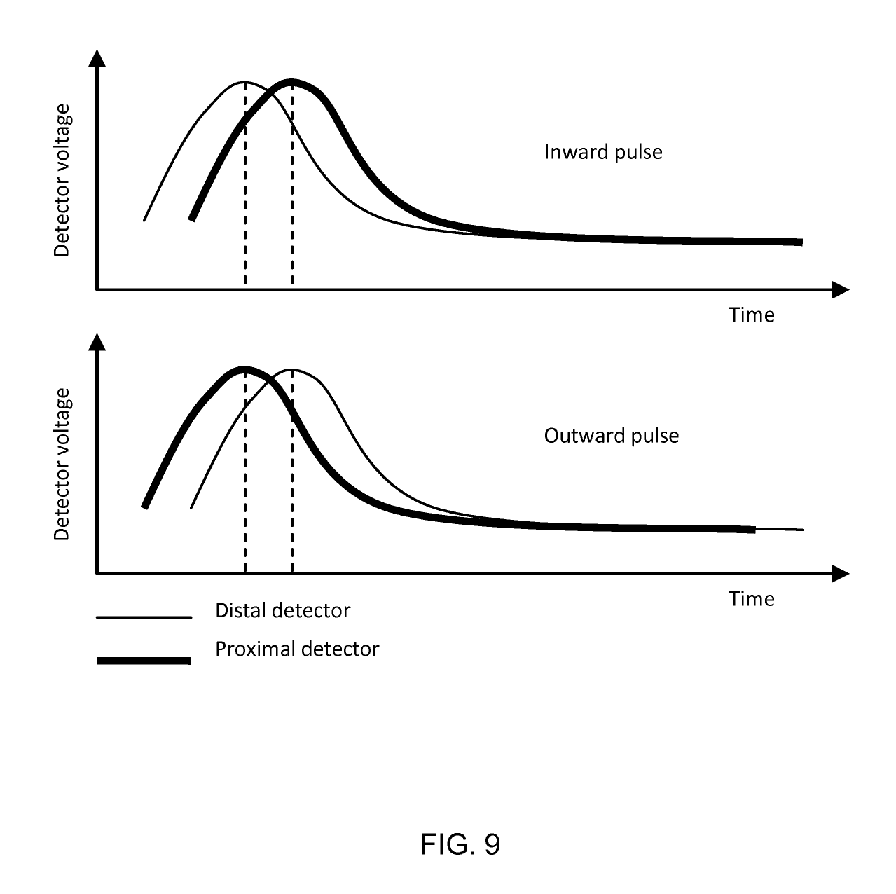

An example of the foregoing effect is depicted notionally in the two plots of FIG. 9. The top graph in FIG. 9 is a plot of detector output voltage versus time for an “inward pulse,” i.e., a pulse traveling away from the subject's extremities, and shows the output voltage from a detector in a first array (e.g., array 801) and the output voltage from a detector in a second array (e.g., array 803). The bottom graph in FIG. 9 is a plot of detector output voltage versus time for an “outward pulse,” i.e., a pulse traveling toward the subject's extremities. As with the top graph, the bottom graph shows the output voltage from a detector in a first array (e.g., array 801) and the output voltage from a detector in a second array (e.g., array 803). The difference in time of arrival of the pulse at the two detectors may be determined using any convenient feature of the waveform. For instance, as shown in FIG. 9, such a measurement is illustrated by determining the time at which the peak of the waveform arrives at the first detector and at the second detector, with the time difference being represented by the spacing between the two vertical dashed markers. Such time differences are representative of the wave speed and may be used to reject unusual waves or shocks from the normal pulse count.

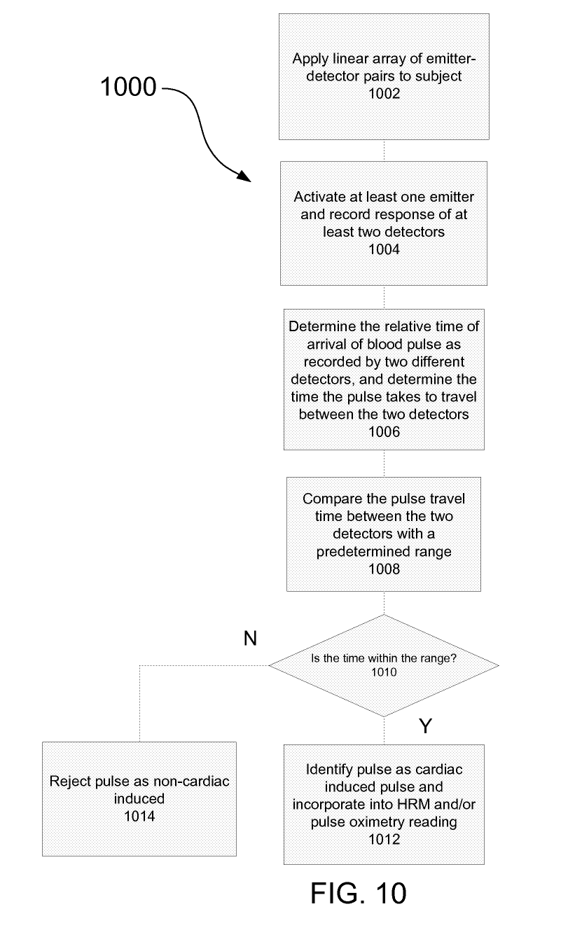

FIG. 10 is a flow chart that depicts a process 1000 for performing identification and rejection of spurious non-cardiac induced pulses that would otherwise be incorrectly reported by HRM or pulse oximetry. In a first step, a device for sensing heart rate and/or performing pulse oximetry according to the present disclosure is positioned (1002) on or near the subject (e.g., on the subject's limb), in which the device includes at least two light detectors and at least one light emitter source. The detectors are arranged with respect to one another in a direction generally aligned with the prevailing blood flow, and each detector is capable of sensing the occurrence and time of arrival of a blood pulse, and of communicating this information electronically to an electronic processor. The at least one light emitter and detectors are activated (1004). The signals measured by the at least two detectors are transmitted to the electronic controller, which, in turn, determines (1006) the relative time of arrival of the blood pulses at the different detectors. The processor then compares (1008) the time required to travel between the two detectors with a pre-determined acceptable range and determines whether the pulse is acceptable or unacceptable (1010). Should the measured value fall outside of the pre-determined acceptable range, the measured pulse is rejected (1014) as an unacceptable non-cardiac induced pulse. Otherwise, the pulse is identified as an acceptably cardiac induced pulse or likely to be cardiac induced pulse (1012). The acceptable pulses then are incorporated into a heart rate or pulse oximetry reading. Determination of the relative time of arrival in (1006) may be carried out using either analog or digital electronics in the electronic controller. For instance, in an implementation that utilizes analog electronics, a differential amplifier may be used, in which one input to the amplifier is from a first light detector (e.g., a light detector nearest to a subject's extremities) and the other input to the amplifier is from a second light detector (e.g., a light detector furthest from the subject's extremities). The occurrence of two nearly synchronous rising (or falling) transients will produce a characteristic pulse at the output of the differential amplifier, in which the amplitude and/or width of the amplifier output pulse may be used in conjunction with a comparator to identify cardiac induced or non-cardiac induced blood pulses. It should be noted that any of the steps discussed above with respect to FIG. 4 (e.g., selecting the most appropriate emitter-detector combination) may also be included in the process 1000.

Configurations of the emitter-detector sensor arrays for identifying cardiac induced and non-cardiac induced pulses other than the arrangement shown in FIG. 8 are also possible. For instance, in some implementations, in place of two emitter-detector arrays, the device may include two separate rows of light detectors arranged on either side of and aligned with a single row of light emitting elements (e.g., one row of detecting elements arranged on the downstream side of the blood flow and the other row of detecting elements arranged on the upstream side of the blood flow). Thus, light from one emitter element may reach and be utilized by two different detector elements: one detector from the upstream row and one detector from the downstream row. In such an implementation, the best emitter-detector combination may still be determined independently.

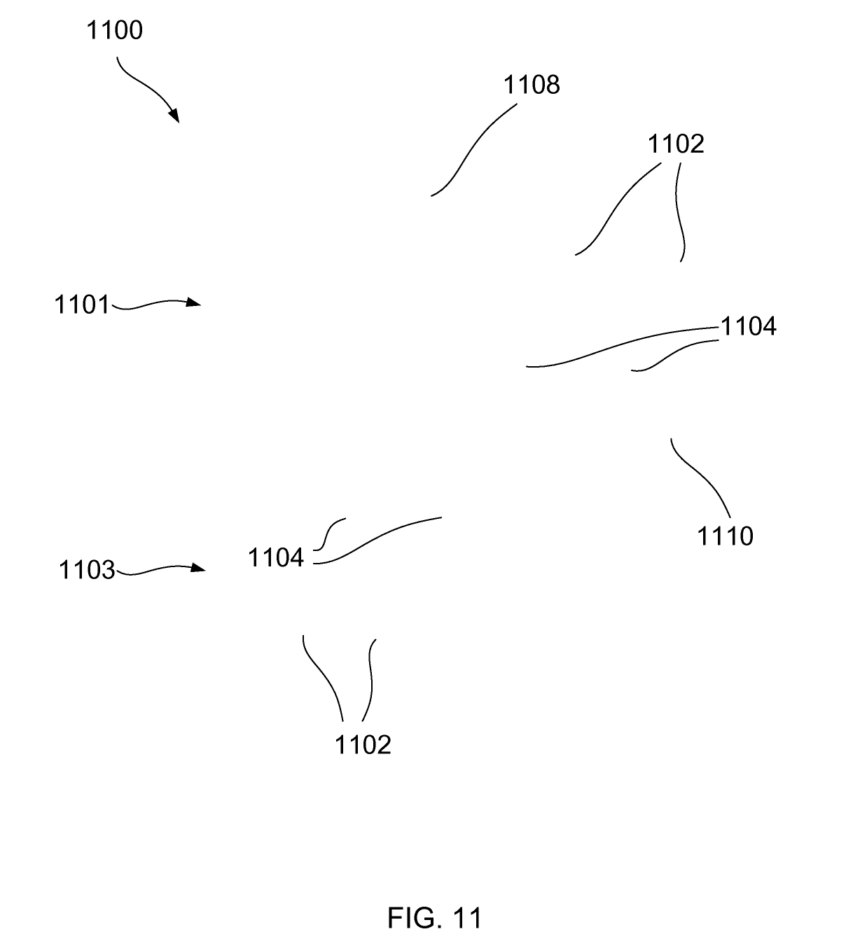

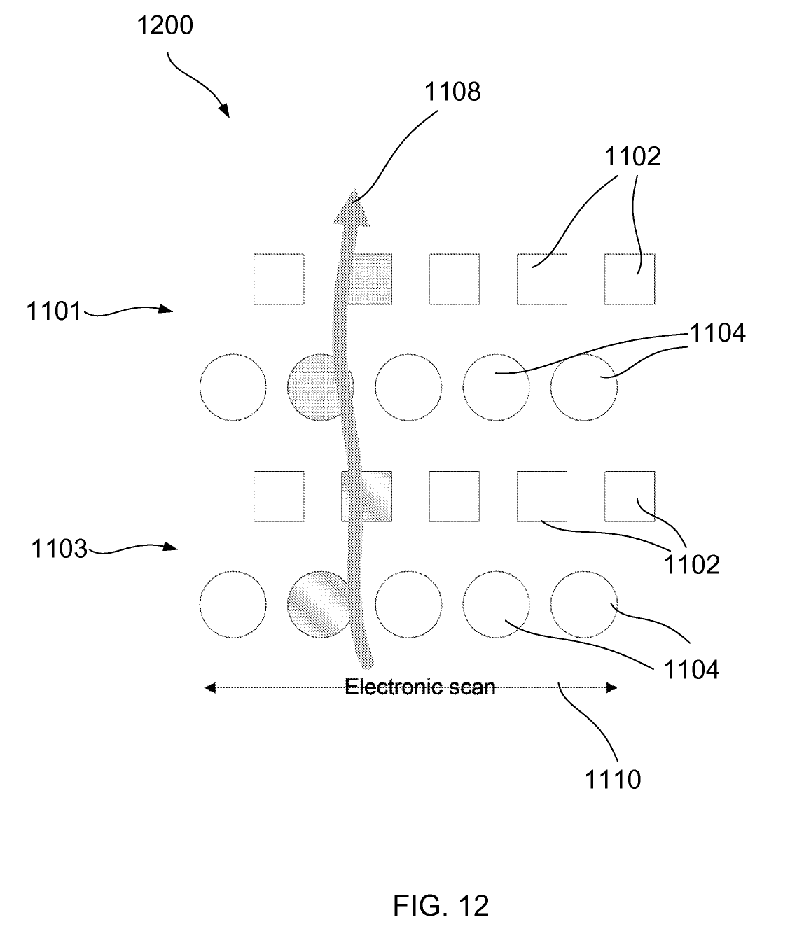

In some implementations, the row of light emitting elements may be staggered with respect to the light detecting elements in each array. For example, FIG. 11 is a schematic that illustrates a configuration in which a first array 1101 includes a row of light detectors 1102 shifted relative to a row of light emitters 1104 by about ½ the pitch between adjacent detectors 1102. Similarly, a second array 1103 includes a row of light detectors 1102 shifted relative to a row of light emitters 1104 by about ½ the pitch between adjacent detectors 1102. The active emitter-detector combinations in FIG. 11 are shown as being filled with a hatched pattern or being filled with a dot pattern. The emitter-detector combinations from the two arrays may operate at different wavelengths or the same wavelength of light. The configuration in FIG. 11 improves the probability that light from the emitters will impinge on an arterial vessel 1108 and be detected. As in other configurations, possible scan directions 1110 for activating the light emitters and detectors are identified. Other convenient cyclic scanning also may be employed. FIG. 12 is an alternative configuration 1200 for the implementation shown in FIG. 11. The reference numerals in FIG. 12 represent the same features as they do in FIG. 11. Again, the active emitter-detector combinations in FIG. 12 are shown as being filled with a hatched pattern or being filled with a dot pattern. The emitter-detector combinations from the two arrays may operate at different wavelengths or the same wavelength of light.

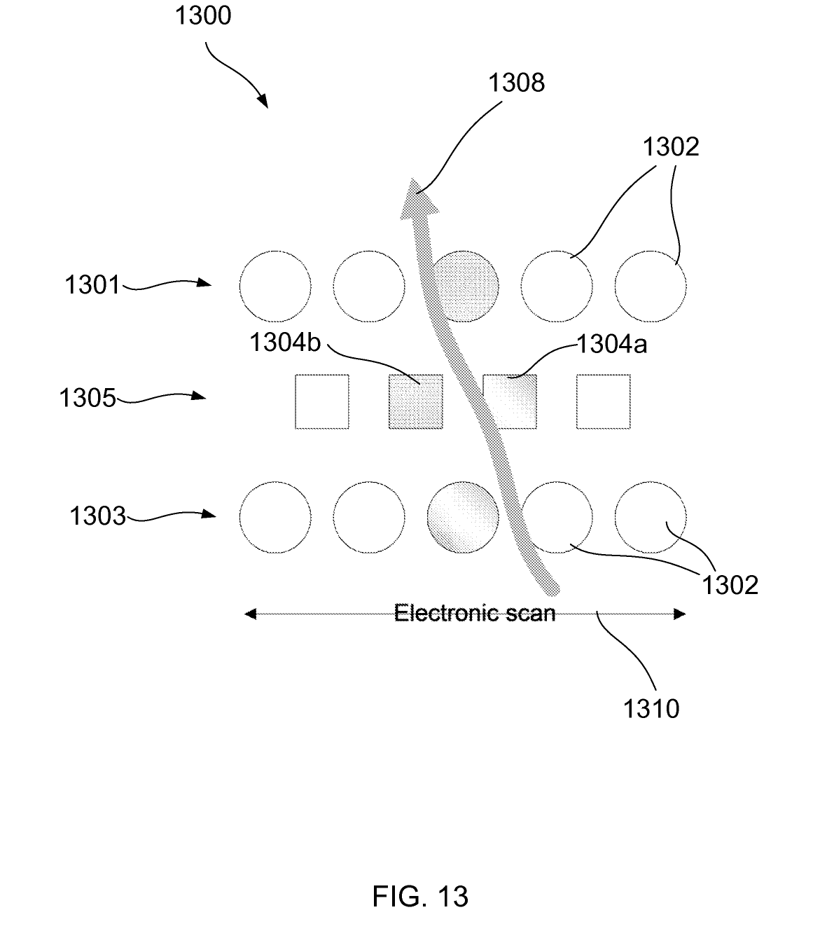

FIG. 13 is a schematic that illustrates another example configuration that improves the probability that light from the emitters will impinge on the arterial vessel 1308 and be detected. The device 1300 includes a first row 1301 of detectors 1302, a second row 1303 of detectors 1302, and a row 1305 of emitters 1304 that is shared by the two detector rows. As shown in FIG. 13, the first and second detector rows 1301, 1303 are shifted relative to the row 1305 of emitters 1304 by about ½ the pitch between adjacent detectors 1302. By reducing the number of rows of emitters, the device 1300 further reduces the complexity of the device, the potential power consumption, and cost (i.e., through fewer parts). Again, the active emitter-detector combinations in FIG. 13 are shown as being filled with a hatched pattern or being filled with a dot pattern. Alternating emitters 1304 (e.g. 1304a and 1304b) in FIG. 13 may operate at different wavelengths or at the same wavelength of light. Similarly, the detectors 1302 may be configured to sense different wavelengths of light corresponding to different light wavelengths generated by the emitters 1304 or each detector 1302 may be configured to sense the same wavelength of light.

Heart Rate Monitoring with Plethysmographic Discrimination

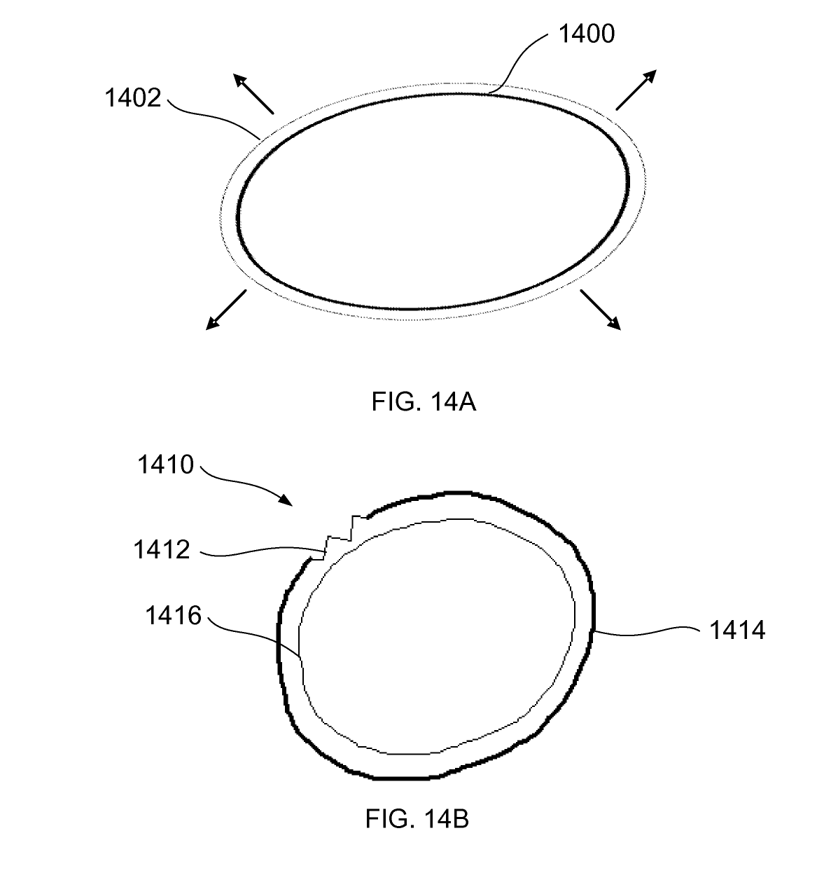

Plethysmography is the measuring of changes in volume within an organ or body resulting from fluctuations in the amount or the pressure of blood or air contained in the organ or body. The devices encompassed by the present disclosure may be configured to operate as plethysmographic devices by detecting the volumetric and pressure waves associated with a blood pulse. An example configuration of a device for performing plethysmographic HRM is shown in FIG. 14B. FIG. 14A is a schematic that illustrates an exaggerated and approximate expansion of a human limb due to the effect of a volumetric fluid wave arriving at a particular cross section of the limb in the absence of a lateral movement. In particular, the schematic in FIG. 14A shows a cross-section 1400 of a limb at a first time and a cross-section 1402 of the same limb at a second time after it has expanded as a result of a pressure pulse through arterial blood vessels in the limb. The arrows in FIG. 14A indicate the direction of expansion. A more realistic depiction would have the circumferential displacement occur non-uniformly around the limb cross section. The approximation implicit in the figure, however, does not materially affect the operation of the device.

The change in circumference of the limb may be determined by using a number of detector elements generally distributed around the limb, in which the detector elements are arranged on a carrier (e.g., a physical bracelet, band, string, tape or watchband that can be placed around a subject's wrist or ankle) The detector elements may include, for example, strain gauges or an array of accelerometers. For instance, in some implementations, a piece of string or tape may be wrapped around the limb, so that it may be deflected by the radially moving circumference of the limb due to the arterial pressure pulses. The stretching of the circumference then might be detected by one or more strain gauges arranged in line with the string or tape. An electronic controller electronically coupled to the strain gauges may convert the information relating to a change in strain into information relating to a change in circumference. FIG. 14B is a schematic illustrating a cross-section of a user's limb 1416 over which an example of a device 1410 for performing plethysmographic HRM is placed. The device 1410 includes a strain gauge 1412 on a band 1414. The band 1414 is placed around the limb 1416 of the user. The linear dimension of the gauge, multiplied by the measured strain may directly represent the change in circumference of the limb 1416 as the limb 1416 expands and contracts.

Regardless of the method used to sense the change in circumference, the carrier and the corresponding detector(s) act as massive mechanical loads. In the presence of lateral acceleration of the limb (as, for instance, in the case of a runner's swinging forearm), the carrier would undergo some average lateral displacement with a magnitude and direction dependent on mechanical properties of the limb and carrier, as well as on the magnitude and direction of the limb's acceleration.

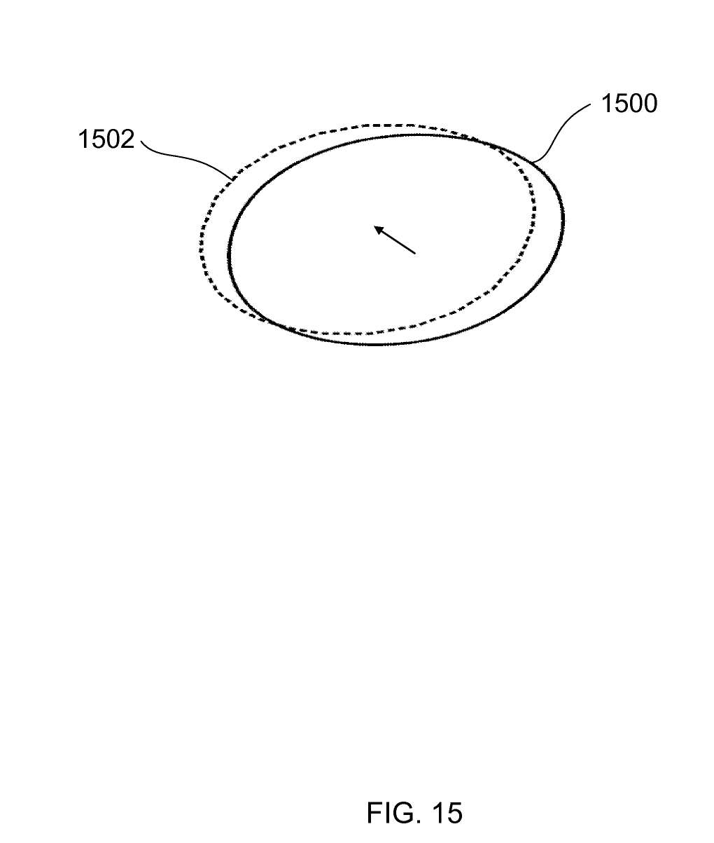

FIG. 15 is a schematic that illustrates the cross-section of a limb before 1500 and after 1502 pure limb movement, without any internal fluid flow. The techniques for determining the expansion of the limb disclosed herein discriminate/differentiate between the motion of the limb due to the lateral movement shown in FIG. 15 and the movement of the limb as a result of the fluid flow through the limb as illustrated in FIG. 14a. In particular, the techniques distinguish between co-moving and oppositely moving points located on opposite ends of a given cross-sectional diameter of the limb being analyzed. For instance, in some implementations, a pair of accelerometers may be positioned on a band at opposite ends of at least two non-collinear diameters of the limb's cross section.

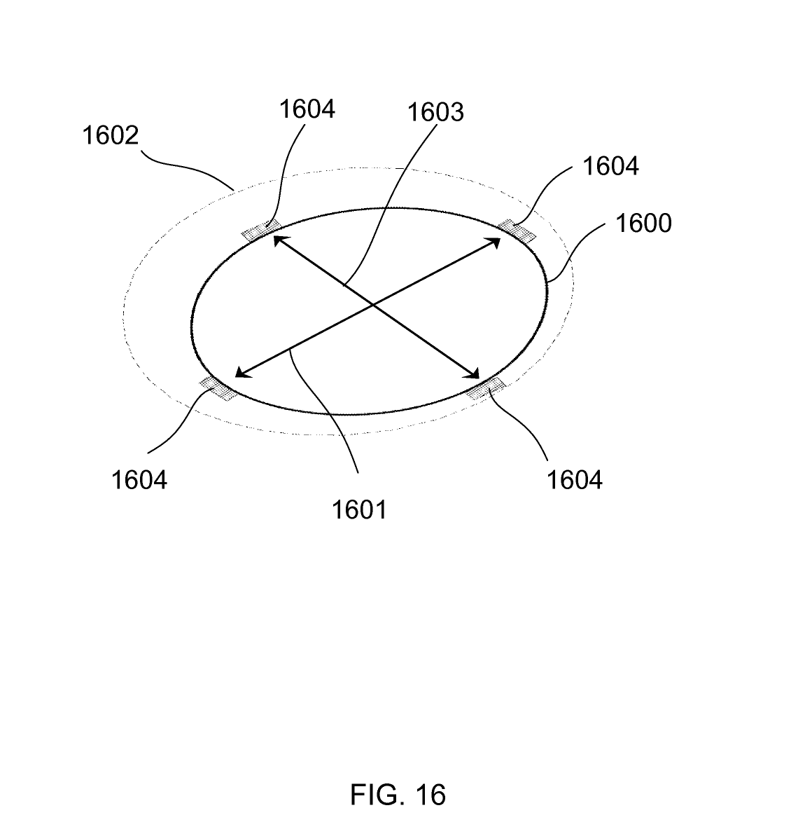

FIG. 16 is a schematic that illustrates an example of such a configuration. As shown in FIG. 16, four accelerometers 1604 are arranged at opposite ends of two non-collinear diameters (indicated by arrows 1601, 1603) of a limb cross-section 1600. The limb cross-section 1600 corresponds to when there is no expansion or lateral movement of the limb. Limb cross-section 1602, which is illustrated using dashed lines, is representative of the limb cross-section after expansion and lateral movement of the limb. The accelerometers 1604 may be positioned on or embedded in a carrier (omitted from FIG. 16 for clarity) such as a bracelet or band or other suitable carrier. The accelerometers 1604 are electronically coupled to an electronic processor (e.g., such as an electronic processor shown in FIG. 3) that may also be positioned on or embedded in the carrier. The electronic processor analyzes the acceleration data measured by the accelerometers and separates the signals from opposite ends of a diameter into differential and common mode components.

Acceleration sensors located at opposite ends of a cross sectional diameter are suitable for detecting whether the portions of the carrier (and thus the limb) adjacent to the sensors have moved radially outward or inward by the same amount. If the signals measured by the accelerometers indicate that the adjacent portions of the limb have moved radially outward by the same amount, this corresponds to a pure volumetric expansion of the limb. If the signals measured by the accelerometers indicate that the adjacent portions of the limb have moved radially inward by the same amount, this corresponds to a pure contraction of the limb. Whether the accelerometers measure radially outward or inward movement depends on the sign and magnitude of the signal being measured. For instance, each accelerometer may be oriented such that the positive acceleration direction corresponds to a direction along the outward normal to the skin. In those cases, radially outward movement of the limb skin would be associated with positive accelerometer signals and radially inward movement of the limb would be associated with negative accelerometer signals. Purely lateral movement of the limb corresponds to scenarios where one of the sensors records negative movement while the other opposite sensor records positive movement. Of course, the signals measured by the accelerometers also may correspond to combined lateral and radial expansion/contraction of the limb. Preferably, the imaginary lines connecting opposing acceleration sensors pass through the approximate center of gravity of the limb's cross section. Furthermore, it is preferable that the lines intersect the circumference of the limb cross section such that the intersection points on the circumference are distributed as uniformly as possible, so that an accurate analysis of the limb movement may be obtained.

Measurement signals from the accelerometers may be coupled to the inputs of differential amplifiers. The differential amplifiers may be incorporated into an electronic processor, such as the processor 306 shown in FIG. 3, or may be standalone differential amplifiers coupled to the electronic processor. The pair of signals from two acceleration sensors arranged at opposite ends of a limb cross sectional diameter may be decomposed by the differential amplifiers into a common mode portion (corresponding to their average value) and a differential portion (corresponding to their difference). The differential portion is indicative of lateral acceleration (i.e., movement of the carrier, and thus the user's limb) while the common mode signal primarily is related to volumetric expansion/contraction of the carrier (and thus limb), which is indicative of plethysmographic information including the user's heart rate. Thus, the electronic processor may analyze the common mode signal to determine a user's heart rate.

If the acceleration is not aligned with either of the sensor pair diameters, the signals are decomposed vectorially. For example, in the case of two pairs of sensors such as shown in FIG. 16, the vector decomposition of the signals is performed (a) along the cross sectional diameters corresponding to the two pairs of sensors, and (b) into the differential and common mode components, as discussed above. These signal components may be used alone or in combination with other sensor types as discussed above. For instance, the strain gauge in series with the circumferential band may be used to provide additional plethysmographic information. Alternatively, two of the accelerometers may be replaced by the strain gauge in series with the circumferential band. There should be at least two non-collinear accelerometers to detect at least two lateral acceleration components, in addition to the plethysmographic measurement. The plethysmographic devices disclosed above may also be combined with any of the HRM and pulse oximetry implementations described above.

The components for performing plethysmography and pulse oximetry as described herein, such as the light emitting elements, light detecting elements, accelerometers, electronic processors, power supply and differential amplifiers, among other components, may be incorporated into a single enclosure that is attached to a carrier (e.g., strap, band, bracelet, watchband, tape, or other appropriate structure) for placing on or around a user's limb. In some implementations, the enclosure includes a display (e.g., an light emitting diode display or liquid crystal display) for outputting information, such as O₂ saturation and/or heart rate. In some implementations, the device may additionally or alternatively include other output features for obtaining the information, such as a data port (e.g., universal serial bus, Bluetooth transceiver, an infrared data port, among others).

A number of implementations have been described. Nevertheless, it will be understood that various modifications may be made without departing from the spirit and scope of the invention. For instance, modifications to the relative size, placement, or number of components, and to scanning frequency, light wavelength, or power supplied, among other features, may be made to any of the devices and/or methods disclosed herein. Additionally, the different implementations described herein relating to heart rate monitoring, pulse oximetry, rejection of non-cardiac induced blood pulses, identification of cardiac induced blood pulses, and/or plethysmography may be combined in various different combinations and using different device arrangements, such as any of the arrangements disclosed herein. Other implementations are within the scope of the claims.

1. A device for monitoring arterial blood flow, the device comprising:
a carrier substrate configured to be positioned on or affixed to a user's body;
an array of light emitting elements on the carrier substrate, wherein each light emitting element in the array is arranged to emit light into the user when the carrier is positioned on or affixed to the user's body;
an array of light detecting elements on the carrier substrate, wherein each light detecting element is arranged to detect light generated by one or more of the light emitting elements after the light has reflected from an object within the user's body; and
an electronic controller configured to:
discretely activate one or more of the light emitting elements while simultaneously deactivating one or more other light emitting elements in the array,
receive a signal generated by at least one light detecting element in response to detecting light, and
determine, based on the received signal, a condition of the user's body.

2. The device of claim 1 wherein the condition is a heart rate of the user.

3. The device of claim 1 wherein the condition is a blood oxygenation level of the user.

4. The device of claim 1 wherein the light emitting elements are arranged in a row with approximately equal spacing between adjacent light emitting elements, and wherein the light detecting elements are arranged in a row with approximately equal spacing between adjacent light detecting elements, with the rows approximately parallel to, and laterally offset from each other.

5. The device of claim 4, wherein the row of light emitting elements and the row of light detecting elements are offset both laterally and longitudinally from each other.

6. The device of claim 1 wherein the carrier comprises a watchband.

7. The device of claim 1 wherein the electronic controller comprises a power supply and an electronic processor electronically coupled to the array of light emitting elements and to the array of light detecting elements.

8. The device of claim 1 wherein each light detecting element in the array of light detecting elements is aligned with a corresponding light emitting element in the array of light emitting elements, such that each light detecting element is closest to the corresponding light emitting element.

9. The device of claim 1 wherein the array of light emitting elements is laterally and longitudinally offset from the array of light detecting elements, such that each light detecting element is positioned approximately equidistant from two light emitting elements.

10. The device of claim 1 wherein the array of light emitting elements comprises two or more rows of light emitting elements.

11. The device of claim 1 wherein the array of light detecting elements comprises two or more rows of light detecting elements.

12. The device of claim 1, wherein the array of light emitting elements comprises two or more rows of light emitting elements, wherein the array of light detecting elements comprises two or more rows of light detecting elements,
wherein each light emitting element in a first row of light emitting elements is aligned with a corresponding light detecting element in a first row of light detecting elements to form a first emitter-detector row pair, wherein each light emitting element in a second row of light emitting elements is aligned with a corresponding light detecting element in a second row of light detecting elements to form a second emitter-detector row pair, and wherein the first emitter-detector row pair is laterally and longitudinally offset from the second emitter-detector row pair.

13. The device of claim 12, wherein the lateral offset distance is equal to one half of a pitch between adjacent light emitting elements in the first or second row of light emitting elements or one half of a pitch between adjacent light detecting elements in the first or second row of light detecting elements.

14. The device of claim 10 wherein a number of light emitting elements in the first row of light emitting elements equals a number of light emitting elements in the second row of light emitting elements.

15. The device of claim 14 wherein each light emitting element in the first row of light emitting elements is aligned with a corresponding light emitting element in the second row of light emitting elements.

16. The device of claim 1 wherein the array of light emitting elements is configured to emit light at a first wavelength and a second different wavelength.

17. The device of claim 10 wherein each light emitting element in a first row of light emitting elements is configured to emit light at the first wavelength, and wherein each light emitting element the second row of light emitting elements is configured to emit light at the second wavelength.

18. The device of claim 10 wherein the array of light detecting elements is arranged midway between the first row of light emitting elements and the second row of light emitting elements.

19-40. (canceled)
